(19) United States
(12) Patent Application Publication
(10) Pub. No.: US 2016/0187316 A1
(43) Pub. Date: Jun. 30, 2016

(54) NOVEL MICROFLUIDIC DEVICES FOR DIAGNOSING RED BLOOD CELLS ABNORMALITIES, AND METHODS USING SAME

(71) Applicants: Alexey Aprelev, Philadelphia, PA (US); Frank Ferrone, Bryn Mawr, PA (US)

(72) Inventors: Alexey Aprelev, Philadelphia, PA (US); Frank Ferrone, Bryn Mawr, PA (US)

(73) Assignee: Drexel University, Philadelphia, PA (US)

(21) Appl. No.: 15/062,981

(22) Filed: Mar. 7, 2016

Related U.S. Application Data
(63) Continuation of application No. PCT/US2014/059680, filed on Oct. 8, 2014.
(60) Provisional application No. 61/888,376, filed on Oct. 8, 2013.
(60) Provisional application No. 61/888,834, filed on Oct. 9, 2013.
(60) Provisional application No. 61/894,730, filed on Oct. 23, 2013.

(51) Int. Cl. G01N 33/49

(52) U.S. Cl. CPC G01N 33/4915

(57) ABSTRACT

The present invention includes a device useful for evaluating the size, shape, surface texture, mobility, rigidity, flexibility, tensile resistance or turnover rate of red blood cells in a blood sample from an individual. The device of the present invention may be used to determine if the individual suffers from a red blood cell abnormality, such as sickle cell disease.

### CROSS-REFERENCE TO RELATED APPLICATIONS

This application is a continuation of International Application No. PCT/US2014/059680, filed Oct. 8, 2014, which claims priority to U.S. Provisional Patent Application Ser. No. 61/888,376, filed Oct. 8, 2013, U.S. Provisional Patent Application Ser. No. 61/888,834, filed Oct. 9, 2013; and U.S. Provisional Patent Application Ser. No. 61/894,730, filed Oct. 23, 2013, all of which applications are hereby incorporated herein by reference in their entireties.

### BACKGROUND OF THE INVENTION

Sickle cell disease is a hereditary blood disorder, characterized by red blood cells that assume an abnormally rigid sickle shape. Sickling decreases the flexibility of the cells and causes serious health complications such as vaso-occlusion, spleen infarction, aplasia, anemia, stroke, gallstones, vascular necrosis, decreased immune reactions, bacterial bone infection, acute papillary necrosis of the kidney, leg ulcers, spontaneous abortion, pre-eclampsia, chronic pain, pulmonary hypertension, capillary blocking and chronic renal failure.

Life expectancy for patients afflicted with the disease is shortened. In 1994, in the U.S., the average life expectancy of persons with this condition was 42 years in males and 48 years in females. Today, thanks to better management of the disease, patients can live into their 70s or beyond. Sickle cell disease occurs more commonly among people whose ancestors lived in tropical and sub-tropical sub-Saharan regions where malaria was endemic. Where malaria is common, humans with one of the two alleles of sickle cell disease (sickle cell trait) show less severe symptoms when infected with malaria.

Sickle cell disease is caused by a point mutation in the β-globin chain of hemoglobin, causing the hydrophilic amino acid glutamic acid to be replaced with the hydrophobic amino acid valine at the sixth position of the β-globin chain. The association of two wild-type α-globin subunits with two mutant β-globin subunits forms hemoglobin S. Under low-oxygen conditions (in the venous circulation, for example), the absence of a polar amino acid at position six of the β-globin chain promotes the non-covalent polymerization (aggregation) of hemoglobin, and this aggregation distorts red blood cells into a sickle shape and decreases their elasticity. Individuals with one copy of the mutated gene display both normal and abnormal hemoglobin in each of their blood cells

The loss of red blood cell elasticity is central to the pathophysiology of sickle cell disease. Normal red blood cells are quite elastic, and may deform in order to pass through capillaries. In sickle cell disease, low-oxygen tension promotes red blood cell sickling. As a consequence, the rigid blood cells become lodged as they pass through narrow vessels, leading to occlusion and ischemia.

The anemia in sickle cell anemia is caused by hemolysis (destruction of the red cells) because of their misshape. Although the bone marrow attempts to compensate by creating new red cells, it cannot match the rate of destruction. Healthy red blood cells typically live 90-120 days, but sickle cells survive only 10-20 days.

There remains a need to develop novel methods of identifying individuals afflicted with a red blood cell abnormality, such as but not limited to sickle cell disease. The present invention fulfills this need.

### BRIEF SUMMARY OF THE INVENTION

In one aspect, the invention provides devices. In another aspect, the invention provides methods of evaluating a property of red blood cells in a blood sample of an individual, wherein the property comprises size, shape, surface texture, mobility, rigidity, flexibility, tensile resistance or turnover rate. In yet another aspect, the invention provides kits comprising a device of the present invention, an applicator, and an instructional material for use thereof.

In certain embodiments, the device comprises a central tube with two open extremities. In other embodiments, each extremity of the central tube is attached to a terminal open capillary tube. In yet other embodiments, the central tube is at least partially filled with a filling selected from the group consisting of microspheres, hydrogel, synthetic fibers, and any combinations thereof.

In certain embodiments, the two terminal open capillary tubes have about the same inner diameter. In other embodiments, the two terminal open capillary tubes do not have about the same inner diameter. In yet other embodiments, the two terminal open capillary tubes and the central tube are physically fused as to form a single unit. In yet other embodiments, the central tube has about the same inner diameter as the two terminal open capillary tubes. In yet other embodiments, the central tube has a larger inner diameter than either of the two terminal open capillary tubes.

In certain embodiments, the filling is at least partially infused with a chemical agent that promotes deoxygenation or adhesion of a red blood cell. In other embodiments, the chemical agent comprises a dithionite salt, an ascorbate salt, or a sulfite salt.

In certain embodiments, the method optionally comprises pre-rinsing the device of the present invention with a chemical agent that promotes deoxygenation or adhesion of a red blood cell. In other embodiments, the method optionally comprises pre-rinsing the device of the present invention with a blood sample of an individual. In yet other embodiments, the method comprises contacting a blood sample of an individual with the device of the present invention, whereby the blood sample penetrates one of the terminal open capillary tubes. In yet other embodiments, the method comprises measuring the flow rate of the blood sample through the device. In yet other embodiments, the method of the present invention allows evaluating the property of the red blood cells in the blood sample.

In certain embodiments, measuring the flow rate comprises measuring the time required for the blood sample to flow between a set of pre-scored marks on one of the terminal open capillary tubes.

In certain embodiments, the method further comprises measuring the flow rates for at least one selected from the group consisting of: (a) a blood sample of a subject afflicted with a red blood cell abnormality, or a synthetic sample with about the same flow rate as the blood sample of a subject afflicted with a red blood cell abnormality; and (b) a blood sample of a subject not afflicted with a red blood cell abnormality, or a synthetic sample with about the same flow rate as the blood sample of a subject not afflicted with a red blood cell abnormality; thereby generating a calibration curve that correlates flow rate with abnormality state.

In certain embodiments, the calibration curve is used to evaluate whether the individual is afflicted with the red blood cell abnormality. In other embodiments, the calibration curve is used to evaluate the severity of the individual's red blood cell abnormality.

In certain embodiments, the red blood cell abnormality comprises at least one selected from the group consisting of a blood disorder, a red blood cell infection, and a disease or disorder that causes altered blood flow. In other embodiments, the blood disorder includes leukemia or anemia. In yet other embodiments, the red blood cell infection includes malaria parasite infection. In yet other embodiments, the disease or disorder includes sickle cell disease, hypertension, or diabetes mellitus.

In certain embodiments, the instructional material comprises instructions for evaluating whether an individual is afflicted with a red blood cell abnormality. In other embodiments, the instructional material recites that the flow rate of the blood or fractions thereof of the individual is compared with at least one selected from the group consisting of: (a) a blood sample of a subject afflicted with a red blood cell abnormality, or a synthetic sample with about the same flow rate as the blood sample of a subject afflicted with a red blood cell abnormality; and (b) a blood sample of a subject not afflicted with a red blood cell abnormality, or a synthetic sample with about the same flow rate as the blood sample of a subject not afflicted with a red blood cell abnormality.

In certain embodiments, the filling of the device of the present invention is at least partially infused with a chemical agent that promotes deoxygenation or adhesion of a red blood cell.

In certain embodiments, the kit further comprises at least one selected from the group consisting of: (a) a blood sample of a subject afflicted with a red blood cell abnormality, or a synthetic sample with about the same flow rate as the blood sample of a subject afflicted with a red blood cell abnormality; and (b) a blood sample of a subject not afflicted with a red blood cell abnormality, or a synthetic sample with about the same flow rate as the blood sample of a subject not afflicted with a red blood cell abnormality.

### BRIEF DESCRIPTION OF THE DRAWINGS

For the purpose of illustrating the invention, there are depicted in the drawings certain embodiments of the present invention. However, the invention is not limited to the precise arrangements and instrumentalities of the embodiments depicted in the drawings.

FIG. 1 illustrates a device of the present invention, with non-limiting illustration of dimension thereof

FIGS. 2A-2C provide a set of graphs illustrating experimental data. FIG. 2A depicts scaled rise times of glycerol solutions, as a function of viscosity, adjusted by fraction of glycerol. Two diameters of tubes were used, but are scaled according to theory. FIG. 2B depicts rise time of oxygenated blood as the original volume is diluted. Dilution lowers viscosity. FIG. 2C depicts rise time for partially deoxygenated blood, illustrating the difference between oxygenated and deoxygenated blood.

FIG. 3 is a set of graphs illustrating optical absorbance spectra of blood measured in 0.25 μL capillaries. The points are measurements, and the solid lines show the fit to known standards. The top spectrum corresponds to oxygenated blood, the center corresponds to 50% oxygenated blood, and the bottom corresponds to deoxygenated blood. In certain embodiments, the relative values allow for estimating and/or determining the fractional oxygen content.

### DETAILED DESCRIPTION OF THE INVENTION

The present invention relates to the unexpected discovery of devices that may be used to identify individuals afflicted with a red blood cell abnormality, such as but not limited to sickle cell disease.

As described herein, a sample of the individual's blood, or fractions thereof, is contacted with a device of the present invention, whereby the blood or fractions thereof flows through the device. In one aspect, the flow rate of the blood or fractions thereof through the device correlates with the size, shape, surface texture, mobility, rigidity, flexibility, tensile resistance or turnover rate of the individual's red blood cells. In another aspect, the flow rate of the blood or fractions thereof through the device indicates whether the individual is afflicted with a red blood cell abnormality, such as but not limited to a blood disorder, a red blood cell infection, or a disease or disorder that causes altered blood flow.

In certain embodiments, the methods of the present invention are inexpensive and easily implemented in the field, and provide a reliable response in a minimum amount of time, so that the afflicted individual may receive the appropriate medical assistance.

### Definitions

As used herein, each of the following terms has the meaning associated with it in this section.

As used herein, unless defined otherwise, all technical and scientific terms generally have the same meaning as commonly understood by one of ordinary skill in the art to which this invention belongs. Generally, the nomenclature used herein and the laboratory procedures in cell culture, textile science, organic chemistry, and peptide chemistry are those well known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more specifically ±5%, even more specifically ±1%, and still more specifically ±0.1% from the specified value, as such variations are appropriate to perform the disclosed methods.

By the term “applicator,” as the term is used herein, is meant any device including, but not limited to, a hypodermic syringe, a pipette, and the like, for using the devices and/or implementing the methods of the present invention.

As used herein, a “fraction” of an original blood sample comprises at least a portion of any red blood cell(s) contained in the original blood sample.

“Instructional material,” as that term is used herein, includes a publication, a recording, a diagram, or any other medium of expression that can be used to communicate the usefulness of the devices of the present invention in a kit. The instructional material of the kit may, for example, be affixed to a container that contains a device of the present invention or be shipped together with a container containing a device. Alternatively, the instructional material may be shipped separately from the container with the intention that the recipient uses the instructional material and a device cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, the term “patient” or “individual” or “subject” refers to a human or a non-human mammal or other living creature with a circulatory system comprising blood.

Non-human mammals include, for example, livestock and pets, such as ovine, bovine, porcine, canine, feline and murine mammals. Other creatures might include fish. In certain embodiments, the patient or subject is human.

As used herein, the term “protein” and any term used to define a specific protein or class of proteins further includes, but is not limited to, fragments, analogs, conservative amino acid substitutions, non-conservative amino acid substitutions and substitutions with non-naturally occurring amino acids with respect to a protein or type or class of proteins. Thus, for example, collagen includes, but is not limited to, fragments, analogs, conservative amino acid substitutions, and substitutions with non-naturally occurring amino acids or residues with respect to any type or class of collagen.

As used herein, the term “red blood cell abnormality” refers to a red blood cell's deviation in at least one property (such as size, shape, surface texture, mobility, rigidity, flexibility, tensile resistance or turnover rate) from the average parameters associated with red blood cells. Such abnormalities may be associated with various diseases, disorders or conditions that cause a red blood cell to change, for example, size, shape, surface texture, mobility, rigidity, flexibility, tensile resistance or turnover rate. Examples of such diseases, disorders or conditions include, but are not limited to, blood disorders (such as, but not limited to, leukemias or anemias), red blood cell infections (such as, but not limited to, malaria parasite infection), or diseases or disorders that cause altered blood flow (such as, but not limited to, sickle cell disease, hypertension, or diabetes mellitus). The devices and methods of the present invention may be used to identify a red blood cell abnormality, as well as develop treatments and/or therapies to address such an abnormality.

Throughout this disclosure, various aspects of the present invention can be presented in a range format. It should be understood that the description in range format is merely for convenience and brevity and should not be construed as an inflexible limitation on the scope of the present invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.3, and 6. This applies regardless of the breadth of the range.

### Devices

The invention includes a device comprising a central tube with two open extremities, wherein each extremity of the central tube is attached to a terminal open capillary tube. In certain embodiments, the two terminal open capillary tubes have about the same inner diameter. In other embodiments, the two terminal open capillary tubes do not have about the same inner diameter.

In certain embodiments, the central tube and the two terminal open capillary tubes are physically fused as to form a single unit. In other embodiments, the central tube has about the same inner diameter as the two terminal open capillary tubes. In yet other embodiments, the central tube has a larger inner diameter than either of the two terminal open capillary tubes. In certain embodiments, the device may consist of only one tube, for which the term “central tube” might refer to a region of that tube, roughly central in the device.

The central tube may be at least partially filled with at least one filling selected from the group consisting of microspheres, hydrogel, synthetic fibers, sintered porous plug, and any combinations thereof. In certain embodiments, the filling is at least partially infused with a chemical agent that promotes deoxygenation and/or adhesion of a red blood cell. In other embodiments, the chemical agent comprises a dithionite salt, an ascorbate salt, or a sulfite salt.

When contacted with a device of the present invention, a blood sample or fractions thereof may enter the central tube through one of the terminal open capillary tubes. The blood sample or fractions thereof may then flow through the central tube due to capillary forces. The blood sample or fractions may also flow through the central tube because negative or positive pressure is applied to one or both of the extremities of the device.

Without wishing to be limited by any theory, as the blood sample or fractions thereof flows through the device, the red blood cells interact with the filling and move at a rate that is influenced by their size, shape, surface texture, mobility, rigidity, flexibility, tensile resistance or turnover rate.

In certain embodiments, the red blood cells of an individual afflicted with sickle cell disease are more rigid and less flexible than those of a normal individual (i.e., an individual not afflicted with a red blood cell abnormality, such as but not limited to sickle cell disease). In certain embodiments, the red blood cells of an individual afflicted with sickle cell disease flow more slowly through the tube than those of a normal individual. Also in certain embodiments, the red blood cells of an individual afflicted with sickle cell disease will flow more slowly through the tube when deoxygenated (in part or entirely) than when oxygenated.

The flow rate of the blood through the tube may be evaluated using photosensors attached to distinct sections of the central tube and/or terminal capillary tubes. The flow rate of the blood through the tube may also be evaluated through windows (i.e., transparent or semi-transparent sections) that provide a view of the interior of the central tube and/or terminal capillary tubes.

Typical flow rates through the device may be determined using blood or fractions thereof from normal individuals, individuals that are known to have a red blood cell abnormality (such as sickle cell disease), and/or individuals that are known to have distinct degrees of severity of a red blood cell abnormality (such as sickle cell disease), whereby the correlation between the flow rate of blood and the abnormality state is obtained.

In certain embodiments, one skilled in the art identifies or prepares a synthetic solution (which may be blood-free or contain one or more blood fractions) that has about the same flow rate as the blood or fractions thereof from normal individuals, individuals that are known to have a red blood cell abnormality, and/or individuals that are known to have distinct degrees of severity of a red blood cell abnormality. In that case, the synthetic solution may be used as a standard for the devices of the present invention, because its flow rate is about the same as the flow rate for the blood or fractions thereof from normal individuals, individuals that are known to have a red blood cell abnormality, and/or individuals that are known to have distinct degrees of severity of a red blood cell abnormality

In certain embodiments, the flow rate of blood through the device of the present invention allows one to evaluate the size, shape, surface texture, mobility, rigidity, flexibility, tensile resistance or turnover rate of the red blood cells in the sample. In other embodiments, the flow rate of blood through the device of the present invention allows one to evaluate the severity of the individual's red blood cell abnormality. The diagnosis of the severity of the individual's red blood cell abnormality may help a medical specialist determine the most effective or beneficial medical treatment that the individual may receive.

The invention includes a kit comprising a device of the present invention, an applicator, and an instructional material for use thereof. The instructional material comprises instructions for evaluating whether an individual is afflicted with a red blood cell abnormality. The instructional material recites that the flow rate of the blood or fractions thereof of the individual is compared to the flow rate of the blood of a subject afflicted with a red blood cell abnormality or not afflicted with a red blood cell abnormality. In certain embodiments, the filling of the device of the present invention is at least partially infused with a chemical agent that promotes deoxygenation and/or adhesion of a red blood cell. In other embodiments, the kit further comprises a blood sample of a subject afflicted with a red blood cell abnormality or not afflicted with a red blood cell abnormality. In yet other embodiments, the kit further comprises a synthetic sample that has about the same flow rate as a blood sample of a subject afflicted with a red blood cell abnormality or not afflicted with a red blood cell abnormality.

### Methods

The invention includes a method of evaluating a property of red blood cells in a blood sample of an individual, wherein the property comprises size, shape, surface texture, mobility, rigidity, flexibility, tensile resistance or turnover rate. The method comprises optionally pre-rinsing a device of the present invention with a blood sample of an individual. The method further comprises contacting a blood sample of an individual with a device of the present invention, whereby the blood sample penetrates one of the terminal open capillary tubes. The method further comprises measuring the flow rate of the blood sample through the device, whereby the property of red blood cells in the blood sample is evaluated.

In certain embodiments, measuring the flow rate comprises measuring the time required for the blood sample to flow between a set of pre-scored marks on one of the capillary tubes. In other embodiments, the method further comprises measuring the flow rates for a blood sample of a subject afflicted with a red blood cell abnormality (or a synthetic sample with about the same flow rate as this blood sample) and/or a blood sample of a subject not afflicted with a red blood cell abnormality (or a synthetic sample with about the same flow rate as this blood sample), thereby generating a calibration curve that correlates flow rate with abnormality state. In yet other embodiments, the calibration curve is used to evaluate whether the individual is afflicted with the red blood cell abnormality. In yet other embodiments, the calibration curve is used to evaluate the severity of the individual's red blood cell abnormality.

In certain embodiments, the red blood cell abnormality comprises blood disorders (such as, but not limited to, leukemias or anemias), red blood cell infections (such as, but not limited to, malaria parasite infection), or diseases or disorders that cause altered blood flow (such as, but not limited to, sickle cell disease, hypertension, and diabetes mellitus).

In a non-limiting aspect, the present method is more straightforward than the currently available methods for evaluating the flexibility of red blood cells. Such laborious methods include microscopy counting of sickle cells or evaluation of cell contents through centrifugation following lysis, both of which require expensive equipment and are insensitive when the patient is severely anemic.

It is contemplated that any embodiment discussed in this specification may be implemented with respect to any method or composition of the present invention, and vice versa. Furthermore, compositions of the present invention may be used to achieve methods of the present invention.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

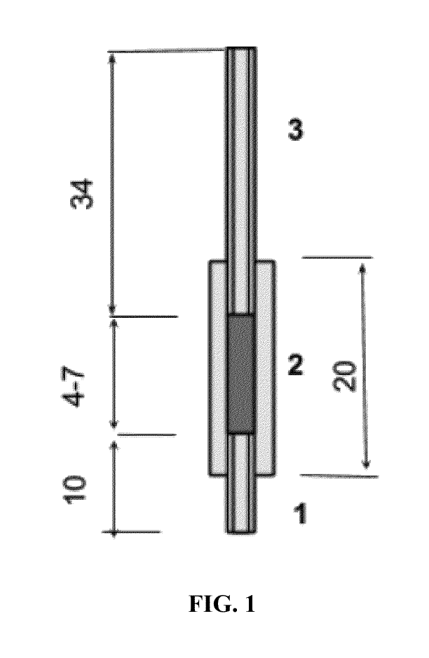

An experiment was performed to test a device of the present invention (illustrated in FIG. 1), including its use in diagnosing sickle cell disease.

Capillary 1 was the probe end, and comprised a DRUMMOND® MICROCAPS® 1 μL pipette (5-10 mm in length). Capillary 2 was the filled capillary, and comprised a DRUMMOND® microdispenser 10 μL (4-7 mm in length). Capillary 3 was the detector capillary, and comprised a DRUMMOND® MICROCAPS® 1 μL pipettes (34 mm in length), with two marks set 10 mm apart from each other.

Capillary 2 was filled with borosilicate solid glass microspheres (130 μm diameter)—COSPHERIC™ BSGMS2.2, Cospheric, Santa Barbara, Calif.

Capillary 3 was open to atmosphere, and could be attached to a standard rubber bulb (not illustrated in FIG. 1).

For the experiment, 15 μL of whole blood were placed in a vial, and the probe end of the device was submerged in the blood. Using a rubber bulb, negative pressure was applied to the device to force the sample into the device, up to the top of the capillary 3. Positive pressure was applied to the device to displace the blood to the start mark in the lower part of the capillary 3. Pressure was then set to zero and a timer was simultaneously started. As blood was pulled into the capillary by surface tension force, flow rate was assessed by measuring the time it took the meniscus to reach the next mark (offset 10 mm above the start mark).

The samples used in the experiment were obtained from two patients. The samples were 100% oxygenated or 0% oxygenated (wherein deoxygenation was achieved by mixing the blood with sodium dithionite).

Using whole blood from Patient No. 1 (30% sickle cells) and a device comprising 7 mm worth of porous filing, the meniscus velocity for oxygenated blood was 2.0 mm/s and for deoxygenated blood was 1.0 mm/s (flow rate ratio of 2 to 1).

Using whole blood from Patient No. 2 (100% sickle cells) and a device comprising 4 mm worth of porous filing, the meniscus velocity for oxygenated blood was 0.5 mm/s and for deoxygenated blood was 0.15 mm/s (flow rate ratio of 3:3 to 1). The accuracy and reproducibility were assessed as ±10%.

### Example 2

The flow rate of normal blood (i.e., blood from an individual who is not afflicted with a red blood cell abnormality) was evaluated using a device of the present invention. Normal blood showed no difference between oxygenated and deoxygenated blood in our device. Under the experimental conditions, the flow time for the oxygenated normal blood was 5.4±1.2 sec, and the flow time for the deoxygenated normal blood was 5.0±0.8 ses (error from triplicate measurement).

In contrast with the normal blood, deoxygenated sickle blood took longer to traverse the porous (bead-filled) region than the oxygenated blood. In one experiment, the flow rate of blood from a patient with only 40% HbS was measured using a device of the present invention. Under the experimental conditions, the patient's oxygenated sickle blood had a rise time of 7.5±4.0 sec, compared to deoxygenated sickle blood times of 30.4±13.7 sec. These averages, as well as all paired comparisons, were outside of each other's error ranges, and thus the presence of sickle cell disease was resolvable in all cases.

### Example 3

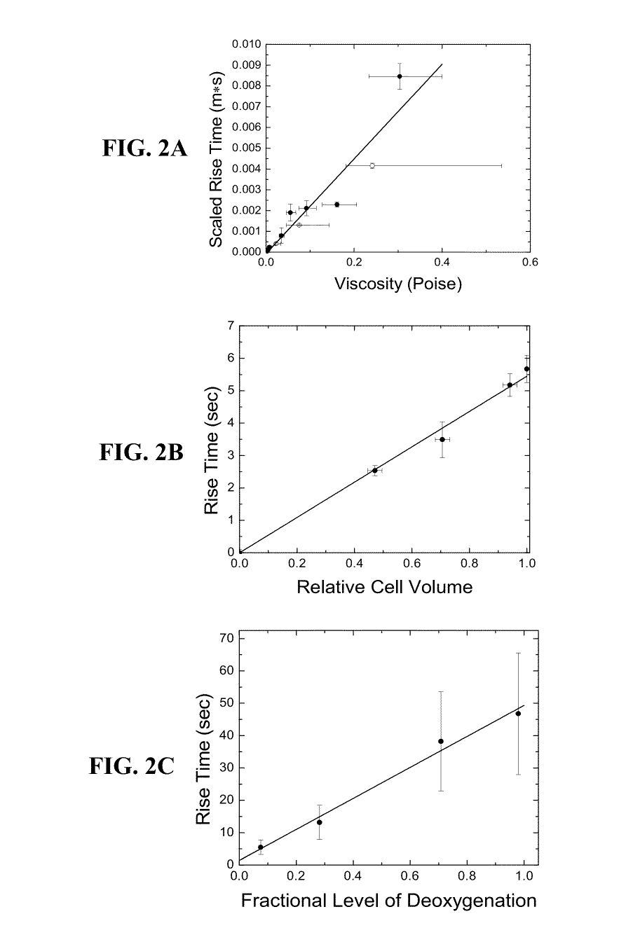

The effect of viscosity on flow rates measured with the devices of the present invention was evaluated. FIG. 2A illustrates rise times on various size capillaries, adjusted for size, as measured for glycerol solutions which viscosity was calculated from standard values. Both theoretical calculations and experimental measurements yielded linear behavior. A similar pattern was observed with a diluted blood sample (FIG. 2B, for oxygenated blood). The relative volume was the result of dilution, with a value of 1.0 corresponding to undiluted blood. As the blood was diluted, the time to transit was reduced, which correlates with its viscosity. The same effect is observed in partially deoxygenated samples (FIG. 1C). Without wishing to be limited by any theory, the viscosity increase in polymerized sickle cells is an important factor in decreasing the flow rate of the corresponding blood sample.

### Example 4

In certain embodiments, the blood sample is deoxygenated before being aspiration into a device of the present invention. In other embodiments, the device (in a non-limiting embodiment, at least a portion of the filling within) is pre-treated with a chemical agent that promotes deoxygenation or adhesion of a red blood cell, before the blood sample is aspirated into the device. In yet other embodiments, at least a fraction of the chemical agent is retained in the filling, even if the filling is rinsed after being pre-treated with the chemical agent. In yet other embodiments, the retained chemical agent contacts at least a portion of the blood sample aspirated into the device.

In one experiment, the device was pre-flushed with a 1 M solution of Na₂S₂O₄, followed by physical expulsion of the dithionite using a rubber bulb for intake and expulsion of the dithionite solution. A few air aspirations were performed to help remove residual solution from the device, and thereby minimize the dilution of the blood sample. In certain embodiments, the blood concentration of dithionite that us required for complete deoxygenation is around 24 mM.

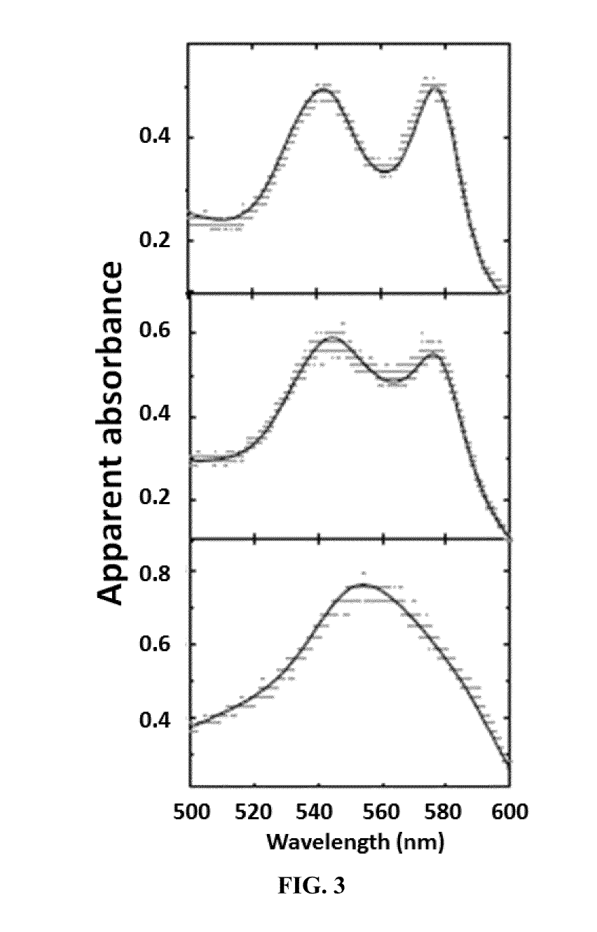

The degree of oxygenation was ascertained by an optical method. An OCEAN OPTICS® fiber optic spectrometer was used to assess oxygenation levels, and a Xenon arc lamp illumination source (with cold mirror) was used to measure the capillary tubes. The experiments allowed for corrections for tube curvature and size, and scattering by the red cells, and representative spectra are illustrated in FIG. 3. The results illustrate the ability of this optical method to evaluate relative amount of oxygenation. FIG. 3 illustrates the obtained spectra (points), fit with standard spectra taken from the literature (solid lines), showing oxygenation and complete deoxygenation. In certain embodiments, constant baseline offset is not relevant within the methods of the present invention.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the present invention. The appended claims are intended to be construed to include all such embodiments and equivalent variations.

1. A device comprising a central tube with two open extremities, wherein each extremity of the central tube is attached to a terminal open capillary tube, wherein the central tube is at least partially filled with a filling selected from the group consisting of microspheres, hydrogel, synthetic fibers, sintered porous plug, and any combinations thereof.

2. The device of claim 1, wherein the two terminal open capillary tubes have about the same inner diameter.

3. The device of claim 2, wherein the central tube has about the same inner diameter as the two terminal open capillary tubes.

4. The device of claim 1, wherein the two terminal open capillary tubes do not have about the same inner diameter.

5. The device of claim 1, wherein the two terminal open capillary tubes and the central tube are physically fused as to form a single unit.

6. The device of claim 1, wherein the central tube has a larger inner diameter than either of the two terminal open capillary tubes.

7. The device of claim 1, wherein the filling is at least partially infused with a chemical agent that promotes deoxygenation or adhesion of a red blood cell.

8. The device of claim 7, wherein the chemical agent comprises a dithionite salt, an ascorbate salt, or a sulfite salt.

9. A method of evaluating a property of red blood cells in a blood sample of an individual, wherein the property comprises size, shape, surface texture, mobility, rigidity, flexibility, tensile resistance or turnover rate, the method comprising:
contacting a blood sample of an individual with a device of claim 1, whereby the blood sample penetrates one of the terminal open capillary tubes, and
measuring the flow rate of the blood sample through the device,
whereby the property of the red blood cells in the blood sample is evaluated.

10. The method of claim 9, further comprising:
pre-rinsing the device with a chemical agent that promotes deoxygenation or adhesion of a red blood cell.

11. The method of claim 9, further comprising:
pre-rinsing the device with a blood sample of an individual,

12. The method of claim 9, wherein measuring the flow rate comprises measuring the time required for the blood sample to flow between a set of pre-scored marks on one of the terminal open capillary tubes.

13. The method of claim 9, further comprising measuring and comparing the flow rates for (a) a blood sample of a subject afflicted with a red blood cell abnormality, which has not been subjected to chemical or physical deoxygenating procedures; and (b) a blood sample of the same subject in which the blood has been deoxygenated wholly or in part, within the device or externally to it.

14. The method of claim 9, further comprising measuring the flow rates for at least one selected from the group consisting of: (a) a blood sample of a subject afflicted with a red blood cell abnormality, or a synthetic sample with about the same flow rate as the blood sample of a subject afflicted with a red blood cell abnormality; and (b) a blood sample of a subject not afflicted with a red blood cell abnormality, or a synthetic sample with about the same flow rate as the blood sample of a subject not afflicted with a red blood cell abnormality; thereby generating a calibration curve that correlates flow rate with abnormality state.

15. The method of claim 14, wherein the calibration curve is used to evaluate whether the individual is afflicted with the red blood cell abnormality.

16. The method of claim 14, wherein the calibration curve is used to evaluate the severity of the individual's red blood cell abnormality.

17. The method of claim 14, wherein the red blood cell abnormality comprises at least one selected from the group consisting of a blood disorder, a red blood cell infection, and a disease or disorder that causes altered blood flow.

18. The method of claim 17, wherein the blood disorder includes leukemia or anemia.

19. The method of claim 17, wherein the red blood cell infection includes malaria parasite infection.

20. A kit comprising:
a device of claim 1,
an applicator, and
an instructional material for use thereof,
wherein the instructional material comprises instructions for evaluating whether an individual is afflicted with a red blood cell abnormality,
wherein the instructional material recites that the flow rate of the blood or fractions thereof of the individual is compared with at least one selected from the group consisting of: (a) a blood sample of a subject afflicted with a red blood cell abnormality, or a synthetic sample with about the same flow rate as the blood sample of a subject afflicted with a red blood cell abnormality; and (b) a blood sample of a subject not afflicted with a red blood cell abnormality, or a synthetic sample with about the same flow rate as the blood sample of a subject not afflicted with a red blood cell abnormality.
